(19) United States
(12) Patent Application Publication
(10) Pub. No.: US 2025/0009653 A1
(43) Pub. Date: Jan. 9, 2025

(54) PROPELLANTS FOR ANTICHOLINERGIC AGENTS IN PRESSURIZED METERED DOSE INHALERS

(71) Applicant: KINDEVA DRUG DELIVERY L.P., Woodbury, MN (US)

(72) Inventors: Philip COCKS, West Bridgford (GB); Sarah WRIGGLESWORTH, Loughborough (GB); Connor MCCORMACK, Nottingham (GB); James LISTER, Nottinghamshire (GB); Lauren HARRISON, Loughborough (GB); Chad HARALDSON, Apple Valley, MN (US); Joseph MILLER, Oakdale, MN (US); James DRAKE, Hudson, WI (US); Stephen STEIN, Lino Lakes, MN (US)

(21) Appl. No.: 18/689,579

(22) PCT Filed: Sep. 8, 2022
(86) PCT No.: PCT/US2022/042959

§ 371 Date: Mar. 6, 2024

Related U.S. Application Data
(60) Provisional application No. 63/241,677, filed on Sep. 8, 2021.
(60) Provisional application No. 63/315,337, filed on Mar. 1, 2022.
(60) Provisional application No. 63/328,120, filed on Apr. 6, 2022.

(51) Int. Cl. A61K 9/00; A61K 9/08; A61K 31/40; A61K 31/439; A61K 31/5386; A61K 47/06; A61K 47/10

(52) U.S. Cl. CPC A61K 9/008; A61K 9/08; A61K 31/40; A61K 31/439; A61K 31/5386; A61K 47/06; A61K 47/10

(57) ABSTRACT

A composition comprising a solution comprising an active pharmaceutical ingredient comprising an anticholinergic agent, such as ipratropium bromide, and a propellant comprising HFA-152a, HFO-1234ze(E), or both. A metered dose inhaler comprising a metering valve a canister; and an actuator comprising an actuator nozzle; wherein the canister comprises said composition.

The present application claims priority to U.S. Provisional Application No. 63/241,677, filed Sep. 8, 2021, U.S. Provisional Application No. 63/315,337, filed Mar. 1, 2022, and U.S. Provisional Application No. 63/328,120, filed Apr. 6, 2022, all of which are incorporated herein by reference in their entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or chronic obstructive pulmonary disease (COPD). pMDI devices can include an aluminum canister, sealed with a metering valve, that contains medicament formulation. Generally, a typical current medicament formulation includes one or more medicinal compounds present in a liquefied propellant.

Historically, the propellants in most pMDIs had been chlorofluorocarbons (CFCs). However, environmental concerns during the 1990s led to the replacement of CFCs with hydrofluoroalkanes (HFAs) as the most commonly used propellant in pMDIs. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA-134a (CF₃CH₂F) and HFA-227 (CF₃CHFCHF₃) having stated 100-year GWP values of 1300 to 1430 and 3220 to 3350, respectively.

Various other propellants have been proposed over the years. Hydrofluroroolefins (HFOs) and carbon dioxide (CO₂) have been mentioned as a potential propellant for pMDIs, but no pMDI product has been successfully developed or commercialized using either as a propellant.

### SUMMARY

The present disclosure describes, in one aspect, a composition. The composition includes a solution, the solution including an active pharmaceutical agent and HFA-152a, HFO-1234ze(E), or both. One advantage of such compositions is the low stated GWPs of HFA-152a and HFO-1234ze(E), which are 140 and less than 1, respectively.

The active pharmaceutical ingredient (API) includes an anticholinergic agent. The anticholinergic agent preferably includes a long-acting muscarinic antagonist. In certain embodiments, the anticholinergic agent is selected from ipratropium, tiotropium, aclidinium, umeclidinium, glycopyrronium (also referred to herein as “glycopyrrolate”), a pharmaceutically acceptable salt or ester of any of the listed drugs, or a mixture of any of the listed drugs, their pharmaceutically acceptable salts or their pharmaceutically acceptable esters.

In some embodiments, the solution further includes ethanol. In some embodiments, the solution further includes water. For example, in some embodiments where the solution includes water, the water may be acidified using an acid. In some embodiments, the solution includes an acid. In some embodiments the solution contains citric acid. In another aspect, the present disclosure describes a pMDI (also referred to herein as an MDI or metered dose inhaler). The pMDI includes a metering valve, a canister, an actuator that includes an actuator nozzle. The canister includes any one of the previously described aspects and/or embodiments of the composition.

In one embodiment, a formulation is provided that includes: a propellant including HFO-1234ze(E), HFA-152a, or both, ethanol, an acid and an active pharmaceutical ingredient including tiotropium or a pharmaceutically acceptable salt or ester thereof (e.g., tiotropium bromide), wherein the tiotropium or pharmaceutically acceptable salt or ester thereof is dissolved in the composition to form a solution.

In one embodiment, a formulation is provided that includes: a propellant including HFO-1234ze(E), HFA-152a, or both, ethanol, an acid and an active pharmaceutical ingredient including ipratropium or a pharmaceutically acceptable salt or ester thereof (e.g., ipratropium bromide), wherein the ipratropium or pharmaceutically acceptable salt or ester thereof is dissolved in the composition to form a solution.

In one embodiment, a formulation is provided that includes: a propellant including HFO-1234ze(E), HFA-152a, or both, ethanol, an acid and an active pharmaceutical ingredient including glycopyrronium or a pharmaceutically acceptable salt or ester thereof (e.g., glycopyrronium bromide), wherein the glycopyrronium or pharmaceutically acceptable salt or ester thereof is dissolved in the composition to form a solution.

Herein, the term “comprises” and variations thereof do not have a limiting meaning where these terms appear in the description and claims. Such terms will be understood to imply the inclusion of a stated step or element, or group of steps or elements, but not the exclusion of any other step or element, or group of steps or elements. The phrase “consisting of” means including, and limited to, whatever follows the phrase “consisting of.” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may, or may not, be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Throughout this disclosure, singular forms such as “a,” “an,” and “the” are often used for convenience; singular forms are meant to include the plural unless the singular alone is explicitly specified or is clearly indicated by the context.

As used herein, the term “or” is generally employed in its usual sense including “and/or” unless the content clearly dictates otherwise.

The term “and/or” means one or all of the listed elements or a combination of any two or more of the listed elements.

The phrase “ambient conditions” as used herein, refers to an environment of room temperature (approximately 20° C. to 25° C.) and 30% to 60% relative humidity.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Herein, “up to” a number (e.g., up to 50) includes the number (e.g., 50). Herein, “at least” a number (e.g., at least 50) includes the number (e.g., 50). Herein, “no more than” a number (e.g., no more than 50) includes the number (e.g., 50).

Numerical ranges, for example “between x and y” or “from x to y”, include the endpoint values of x and y. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure; this terminology is not used to mean that these features are present, much less common, in the prior art. Unless otherwise specified, only the Background section of this Application refers to the prior art.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF THE DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

### DETAILED DESCRIPTION

The formulations of the present disclosure are solutions (i.e., solution formulations or solution compositions). That is, the formulations include one or more APIs dissolved in the formulations (i.e., solubilized in the propellant and often a cosolvent and/or other components) to form solutions. Herein, a “solution” is a homogeneous solution that does not have particulate material visible to the unaided human eye.

Solution and suspension formulations are fundamentally different pMDI formulation approaches. Different factors need to be considered when undertaking the development of products using either of these formulation approaches. Accordingly, it is not possible to apply the same knowledge and understanding of suspension formulations to solution formulations. In solutions, solubility of the API in the propellant, and optional cosolvent, is the key consideration. Various strategies can be used to improve solubility via use of additional excipients such as polyethylene glycol or water. Typically, solutions give smaller aerosol particle size distributions than suspensions and are generally more efficient than suspensions, but the overall dose may be limited due to the amount of API that can be solubilized. The use of cosolvents in solution pMDIs influences droplet evaporation rates and can also lead to changes in the resulting solid-state particles formed in the lung, which could impact on pharmacological uptake of the API compared with deposited API from a suspension. Also, APIs are at higher risk of chemical degradation in solution formulations and often require specific formulation strategies, such as the use of stabilizing acids and specific selection of container closure systems to maximize chemical stability. These problems are specific to solutions and any teachings specific to suspensions do not necessarily overcome them.

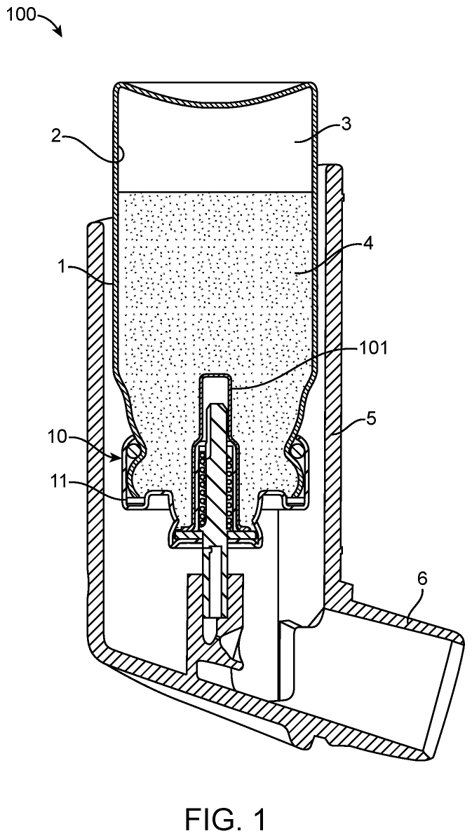

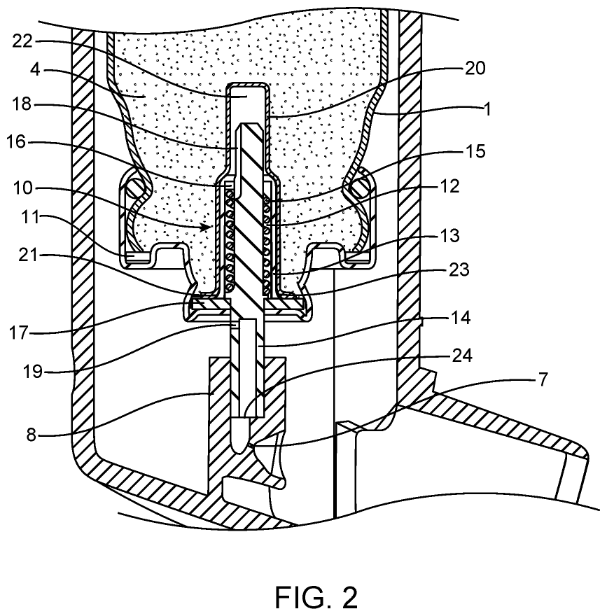

The various embodiments of formulations described herein can be utilized with any suitable inhaler. For example, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister 1 and the ferrule 11 there may be one or more seals. In the embodiments shown in FIGS. 1 and 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port 6 is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol formulation 4 is contained.

The valve 10 shown in FIGS. 1 and 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber 22 besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIGS. 1 and 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

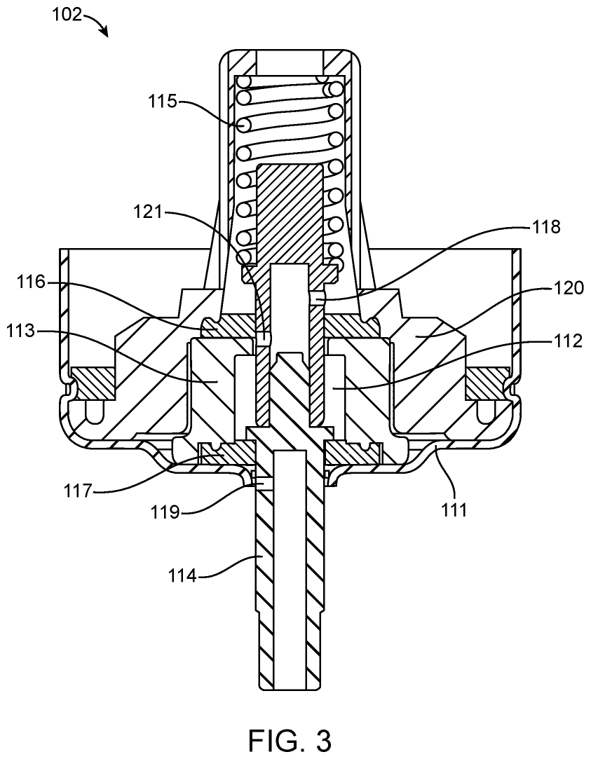

FIG. 3 shows another embodiment of a metered dose aerosol metering valve 102, different from the embodiment shown in FIGS. 1 and 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

The formulation (also called a composition) includes active pharmaceutical ingredient (API) and at least one propellant. In some embodiments, the formulation includes a solvent. In some embodiments, the formulation includes a co-solvent. In some embodiments, the formulation includes one or more additional components (e.g. an acid).

In some embodiments the formulation includes an active pharmaceutical ingredient (API), one propellant, and a solvent. In some embodiments the formulation includes an active pharmaceutical ingredient (API), two propellants, and a solvent. In some embodiments the formulation includes an active pharmaceutical ingredient (API), one propellant, a solvent, and a co-solvent. In some embodiments the formulation includes an active pharmaceutical ingredient (API), two propellants, a solvent, and a co-solvent. In some embodiments the formulation includes an active pharmaceutical ingredient (API), one propellant, a solvent, a co-solvent, and one or more additional components. In some embodiments the formulation includes an active pharmaceutical ingredient (API), two propellants, a solvent, a co-solvent, and one or more additional components (e.g. an acid).

The amount of each component (e.g., active pharmaceutical ingredient, propellant, solvent, co-solvent, and other components) in a given formulation can be described as the wt-% contribution to the total formulation. The “total formulation” or “total composition” refers to all the components included in a given formulation. For example, in some embodiments a formulation may include an active pharmaceutical ingredient, a propellant, a solvent, and a co-solvent. The terms “formulation” and “total formulation” are used interchangeably throughout this disclosure.

Exemplary APIs can include those for the treatment of respiratory disorders, e.g., an anticholinergic agent. Preferably, the anticholinergic APIs can include LAMAs (Long-acting muscarinic antagonists) anticholinergic drugs. Exemplary LAMAs include ipratropium, tiotropium, aclidinium, umeclidinium, glycopyrronium (i.e., glycopyrrolate), a pharmaceutically acceptable salt or ester of any of the listed drugs, or a mixture of any of the listed drugs, their pharmaceutically acceptable salts or their pharmaceutically acceptable esters. In some embodiments, the anticholinergic API is a quaternary ammonium salt, in particular a quaternary ammonium bromide. In some embodiments, the anticholinergic API is selected from ipratropium, tiotropium, glycopyrronium, and a pharmaceutically acceptable salt or ester of any of the listed drugs. In certain embodiments, the API is selected from ipratropium bromide, tiotropium bromide, and glycopyrronium bromide.

In some embodiments, the active pharmaceutical ingredient (API) is ipratropium, a salt thereof, or a hydrate thereof. In some embodiments, the API is ipratropium bromide.

In some embodiments, the API is anhydrous ipratropium bromide. In some embodiments the API is ipratropium bromide monohydrate.

In some embodiments, the API(s) may be dissolved in the composition (e.g., as a solution). In some embodiments, the API is ipratropium, ipratropium bromide, ipratropium bromide monohydrate, and/or anhydrous ipratropium bromide and the API is dissolved in the formulation.

In some embodiments, the active pharmaceutical ingredient (API) is tiotropium, a salt thereof, or a hydrate thereof. In some embodiments, the API is tiotropium bromide. In some embodiments, the API is anhydrous tiotropium bromide. In some embodiments the API is tiotropium bromide monohydrate.

In some embodiments, the API(s) may be dissolved in the composition (e.g., as a solution). In some embodiments, the API is tiotropium, tiotropium bromide, tiotropium bromide monohydrate, and/or anhydrous tiotropium bromide and the API is dissolved in the formulation.

In some embodiments, the active pharmaceutical ingredient (API) is glycopyrronium, or a salt thereof. In some embodiments, the API is glycopyrronium bromide.

In some embodiments, the API(s) may be dissolved in the composition (e.g., as a solution). In some embodiments, the API is glycopyrronium and/or glycopyrronium bromide, and the API is dissolved in the formulation.

The amount of the API in the formulation may vary for each application. For example, the amount of the API in the formulation may be informed by the age, weight, and/or sex of the patient and/or the severity of the disease being treated by the API. In some embodiments, the amount of the API in the formulation may vary based on the number of actuations of the metered dose aerosol valve are needed to deliver a single dose of the pharmaceutically active ingredient.

In some embodiments, the amount of the API is 0.001 wt-% or more, 0.002 wt-% or more, 0.005 wt-% or more, 0.01 wt-% or more, 0.02 wt-% or more, 0.03 wt-% or more, 0.04 wt-% or more, 0.05 wt-% or more, 0.06 wt-% of more, 0.07 wt-% or more, 0.1 wt-% or more, 0.2 wt-% or more, 0.3 wt-% or more, 0.4 wt-% or more, or 0.5 wt-% or more of the total formulation. In some embodiments, the amount of the API is 0.8 wt-% or less, 0.7 wt-% or less, 0.6 wt-% or less, 0.5 wt-% or less, 0.4 wt-% or less, 0.3 wt-% or less, 0.2 wt-% or less, 0.1 wt-% or less, 0.08 wt-% or less, 0.07 wt-% or less, 0.06 wt-% or less, 0.05 wt-% or less, 0.04 wt-% or less, or 0.03 wt-% or less of the total formulation. In some embodiments, the amount of the API is 0.001 wt-% to 0.8 wt-%, 0.01 wt-% to 0.6 wt-%, 0.1 wt-% to 0.5 wt-%, 0.02 wt-% to 0.08 wt-%, 0.02 wt-% to 0.07 wt-%, 0.02 wt-% to 0.06 wt-%, 0.02 wt-% to 0.05 wt-%, 0.02 wt-% to 0.04 wt-%, or 0.02 wt-% to 0.03 wt-% of the total formulation. In some embodiments, the amount of the API is 0.03 wt-% to 0.08 wt-%, 0.03 wt-% to 0.07 wt-%, 0.03 wt-% to 0.06 wt-%, 0.03 wt-% to 0.05 wt-%, or 0.03 wt-% to 0.04 wt-% of the total formulation. In some embodiments, the amount of the API is 0.04 wt-% to 0.08 wt-%, 0.04 wt-% to 0.07 wt-%, 0.04 wt-% to 0.06 wt-%, or 0.04 wt-% to 0.05 wt-% of the total formulation. In some embodiments, the amount of the API is 0.05 wt-% to 0.08 wt-%, 0.05 wt-% to 0.07 wt-%, or 0.05 wt-% to 0.06 wt-% of the total formulation. In some embodiments, the amount of the API is 0.06 wt-% to 0.08 wt-% or 0.06 wt-% to 0.07 wt-% of the total formulation. In some embodiments, the amount of the API is 0.07 wt-% to 0.08 wt-% of the total formulation. In some embodiments, the amount of API is 0.001 wt-% to 0.8 wt-% of the total formulation. In some embodiments, the amount of API is 0.005 wt-% to 0.6 wt-% of the total formulation. In some embodiments, the amount of API is 0.01 wt-% to 0.5 wt-% of the total formulation.

In some embodiments, the amount of ipratropium, ipratropium bromide, ipratropium bromide monohydrate and/or anhydrous ipratropium bromide is 0.02 wt-% or more, 0.03 wt-% or more, 0.04 wt-% or more, 0.05 wt-% or more, 0.06 wt-% of more, or 0.07 wt-% or more of the total formulation. In some embodiments, the amount of ipratropium, ipratropium bromide, ipratropium bromide monohydrate, and/or anhydrous ipratropium bromide is 0.08 wt-% or less, 0.07 wt-% or less, 0.06 wt-% or less, 0.05 wt-% or less, 0.04 wt-% or less, or 0.03 wt-% or less of the total formulation. In some embodiments, the amount of ipratropium, ipratropium bromide, ipratropium bromide monohydrate, and/or anhydrous ipratropium bromide is 0.02 wt-% to 0.08 wt-%, 0.02 wt-% to 0.07 wt-%, 0.02 wt-% to 0.06 wt-%, 0.02 wt-% to 0.05 wt-%, 0.02 wt-% to 0.04 wt-%, or 0.02 wt-% to 0.03 wt-% of the total formulation. In some embodiments, the amount of ipratropium, ipratropium bromide, ipratropium bromide monohydrate, and/or anhydrous ipratropium bromide is 0.03 wt-% to 0.08 wt-%, 0.03 wt-% to 0.07 wt-%, 0.03 wt-% to 0.06 wt-%, 0.03 wt-% to 0.05 wt-%, or 0.03 wt-% to 0.04 wt-% of the total formulation. In some embodiments, the amount ipratropium, ipratropium bromide, ipratropium bromide monohydrate, and/or anhydrous ipratropium bromide is 0.04 wt-% to 0.08 wt-%, 0.04 wt-% to 0.07 wt-%, 0.04 wt-% to 0.06 wt-%, or 0.04 wt-% to 0.05 wt-% of the total formulation. In some embodiments, the amount of ipratropium, ipratropium bromide, ipratropium bromide monohydrate, and/or anhydrous ipratropium bromide is 0.05 wt-% to 0.08 wt-%, 0.05 wt-% to 0.07 wt-%, or 0.05 wt-% to 0.06 wt-% of the total formulation. In some embodiments, the amount of ipratropium, ipratropium bromide, ipratropium bromide monohydrate, and/or anhydrous ipratropium bromide is 0.06 wt-% to 0.08 wt-% or 0.06 wt-% to 0.07 wt-% of the total formulation. In some embodiments, the amount of ipratropium, ipratropium bromide, ipratropium bromide monohydrate, and/or anhydrous ipratropium bromide is 0.07 wt-% to 0.08 wt-% of the total formulation.

Analogous amounts of APIs as listed above for ipratropium apply to other APIs, in particular tiotropium and glycopyrronium, and pharmaceutically acceptable salts or esters thereof.

In embodiments, typical formulations of the present disclosure include the API in an amount of at least 1 μg/actuation, at least 2 μg/actuation, at least 3 μg/actuation, at least 4 μg/actuation, at least 5 μg/actuation, at least 6 μg/actuation, at least 7 μg/actuation, at least 8 μg/actuation, at least 9 μg/actuation, at least 10 μg/actuation, at least 15 g/actuation, at least 25 μg/actuation, at least 30 μg/actuation, at least 40 μg/actuation, at least 50 μg/actuation, at least 60 μg/actuation, at least 70 μg/actuation, at least 80 g/actuation, at least 90 μg/actuation, at least 100 μg/actuation, at least 150 μg/actuation, at least 200 μg/actuation, at least 300 μg/actuation, or at least 400 μg/actuation. In embodiments, typical formulations of the present disclosure include the API in an amount of less than 500 μg/actuation, at most 400 μg/actuation, at most 300 μg/actuation or at most 200 μg/actuation. In some preferred embodiments, formulations of the present disclosure include the API in an amount of 80 μg/actuation to 120 μg/actuation.

The amount of API may be determined by the required dose per inhalation and the pMDI metering valve size, that is, the size of the metering chamber. The size of the metering chamber may be between 5 μL and 200 μL, between 25 μL and 200 μL, between 25 μL and 150 μL, between 25 μL and 100 μL, or between 25 μL and 65 μL.

The formulation (i.e., composition) includes at least one propellant. In some embodiments the propellant includes HFO-1234ze(E), also known as trans-1,1,1,3-tetrafluoropropene, trans-1,3,3,3-tetrafluoropropene, or trans-1,3,3,3-tetrafluoroprop-1-ene. The chemical structure of trans and cis isomers of HFO-1234ze are very different. As a result these isomers have very different physical and thermodynamic properties. The significantly lower boiling point and higher vapor pressure of the trans (E) isomer relative to that of the cis (Z) isomer, at ambient conditions, makes the trans isomer a far more thermodynamically suitable propellant for achieving efficient pMDI atomization.

In some embodiments, the propellant includes HFA-152a, also known as 1,1-difluoroethane. In some embodiments, the composition includes both HFO-1234ze(E) and HFA-152a.

In some embodiments, the amount of HFO-1234ze(E) and/or HFA-152a in the formulation is 70 wt-% or greater, 80 wt-% or greater, 85 wt-% or greater, 90 wt-% or greater, 99 wt-% or greater of the total formulation. In some embodiments, the amount of HFO-1234ze(E) and/or HFA-152a is 99.5 wt-% or less, 99 wt-% or less, 90 wt-% or less, 85 wt-% or less, 80 wt-% or less, or 75 wt-% or less of the total formulation. In some embodiments the amount of HFO-1234ze(E) and/or HFA-152a is 70 wt-% to 99.9 wt-%, 70 wt-% to 99 wt-%, 70 wt-% to 95 wt-%, 70 wt-% to 90 wt-%, 70 wt-% to 85 wt-%, 70 wt-% to 80 wt-%, or 70 wt-% to 65 wt-% of the total formulation. In some embodiments the amount of HFO-1234ze(E) and/or HFA-152a is 75 wt-% to 99.9 wt-%, 75 wt-% to 99 wt-%, 75 wt-% to 95 wt-%, 75 wt-% to 90 wt-%, 75 wt-% to 85 wt-%, or 75 wt-% to 80 wt-% of the total formulation. In some embodiments the amount of HFO-1234ze(E) and/or HFA-152a is 80 wt-% to 99.9 wt-%, 80 wt-% to 99 wt-%, 80 wt-% to 95 wt-%, 80 wt-% to 90 wt-% or 80 wt-% of the total formulation. In some embodiments the amount of HFO-1234ze(E) and/or HFA-152a is 85 wt-% to 99.9 wt-%, 85 wt-% to 99 wt-%, 85 wt-% to 95 wt-%, or 85 wt-% to 90 wt-% of the total formulation. In some embodiments the amount of HFO-1234ze(E) and/or HFA-152a is 90 wt-% to 99.9 wt-%, 90 wt-% to 99 wt-% or 90 wt-% to 95 wt-% of the total formulation. In some embodiments the amount of HFO-1234ze(E) and/or HFA-152a is 95 wt-% to 99.9 wt-%, or 95 wt-% to 99 wt-% of the total formulation. In some embodiments the amount of HFO-1234ze(E) and/or HFA-152a is 99 wt-% to 99.9 wt-% of the total formulation.

In some embodiments, the formulation includes more than one propellant. In some embodiments, the formulation includes two propellants. In some embodiments, the formulation includes three propellants. In some embodiments, the formulation includes four propellants.

In embodiments that have two propellants, the additional propellants may be, for example, a hydrofluoroalkanes such as HFA-134a, HFA-227, HFA-152a, or combinations thereof, hydrofluroroolefins such as HFO-1234yf, HFO-1234ze(E) also known as trans-HFO-1234ze, HFO-1234ze(Z) also known as cis-HFO-1234ze, or combinations thereof. When present, the amounts of the second propellant can be 0.1 wt-% to 20 wt-%, 0.1 wt-% to 5 wt-%, or 0.1 wt-% to 0.5 wt-% of the total composition.

In some embodiments, the formulation includes a solvent. In some embodiments, the solvent is or includes water. In some embodiments, the amount of solvent is 0.1 wt-% or greater, 0.2 wt-% or greater, 0.25 wt-% or greater, 0.3 wt-% or greater, 0.4 wt-% or greater, 0.5 wt-% or greater, 0.6 wt-% or greater, 0.7 wt-% or greater, 0.75 wt-% or greater, 1 wt-% or greater, 5 wt-% or greater, or 10 wt-% or greater of the total formulation. In some embodiments, the amount of solvent is 10 wt-% or less, 5 wt-% or less, 1 wt-% or less, 0.75 wt-% or less, 0.5 wt-% or less, or 0.25 wt-% or less of the total formulation. In some embodiments, the amount of solvent is 0.1 wt-% to 0.25 wt-%, 0.1 wt-% to 0.5 wt-%, 0.1 wt-% to 0.75 wt-%, 0.1 wt-% to 1 wt-%, 0.1 wt-% to 5 wt-%, or 0.1 wt-% to 10 wt-% of the total formulation. In some embodiments, the amount of solvent is 0.25 wt-% to 0.5 wt-%, 0.25 wt-% to 0.75 wt-%, 0.25 wt-% to 1 wt-%, 0.25 wt-% to 5 wt-%, or 0.25 wt-% to 10 wt-% of the total formulation. In some embodiments, the amount of solvent is 0.5 wt-% to 0.75 wt-%, 0.5 wt-% to 1 wt-%, 0.5 wt-% to 5 wt-%, or 0.5 wt-% to 10 wt-% of the total formulation. In some embodiments, the amount of solvent is 0.75 wt-% to 1 wt-%, 0.75 wt-% to 5 wt-%, or 0.75 wt-% to 10 wt-% of the total formulation. In some embodiments, the amount of solvent is 1 wt-% to 5 wt-%, or 1 wt-% to 10 wt-% of the total formulation. In some embodiments, the amount of solvent is 5 wt-% to 10 wt-% of the total formulation.

In some embodiments, the amount of water is 0.1 wt-% or greater, 0.25 wt-% or greater, 0.5 wt-% or greater, 0.75 wt-% or greater, 1 wt-% or greater, 5 wt-% or greater, or 10 wt-% or greater of the total formulation. In some embodiments, the amount of water is 10 wt-% or less, 5 wt-% or less, 1 wt-% or less, 0.75 wt-% or less, 0.5 wt-% or less, or 0.25 wt-% or less of the total formulation. In some embodiments, the amount of water is 0.1 wt-% to 0.25 wt-%, 0.1 wt-% to 0.5 wt-%, 0.1 wt-% to 0.75 wt-%, 0.1 wt-% to 1 wt-%, 0.1 wt-% to 5 wt-%, or 0.1 wt-% to 10 wt-% of the total formulation. In some embodiments, the amount of water is 0.25 wt-% to 0.5 wt-%, 0.25 wt-% to 0.75 wt-%, 0.25 wt-% to 1 wt-%, 0.25 wt-% to 5 wt-%, or 0.25 wt-% to 10 wt-% of the total formulation. In some embodiments, the amount of water is 0.5 wt-% to 0.75 wt-%, 0.5 wt-% to 1 wt-%, 0.5 wt-% to 5 wt-%, or 0.5 wt-% to 10 wt-% of the total formulation. In some embodiments, the amount of water is 0.75 wt-% to 1 wt-%, 0.75 wt-% to 5 wt-%, or 0.75 wt-% to 10 wt-% of the total formulation. In some embodiments, the amount of water is 1 wt-% to 5 wt-%, or 1 wt-% to 10 wt-% of the total formulation. In some embodiments, the amount of water is 5 wt-% to 10 wt-% of the total formulation.

In some embodiments when the solvent is water, the water may be acidified using one or more acids. The acid is used to stabilize a solution pMDI via modification of the hydrogen ion concentration in the formulation in order to minimise drug degradation. In some embodiments, acidified water may facilitate the dissolution of the API in the formulation. In some embodiments, acidified water may increase the stability of the API in a solution formulation. In some embodiments where the API is ipratropium, ipratropium bromide, ipratropium bromide monohydrate, and/or anhydrous ipratropium bromide, acidified water may facilitate the dissolution of the API in the formulation. In some embodiments where the API is ipratropium, ipratropium bromide, and/or anhydrous ipratropium bromide, acidified water may increase the stability of the API in a solution formulation.

Acids that may be used incorporated into the formulations, such as by acidifying the water include, but are not limited to, organic acids such as ascorbic acid, acetic acid, maleic acid, fumaric acid, succinic acid, formic acid, propionic acid, oxalic acid, lactic acid, glycolic acid, or combinations thereof, mineral acids (i.e., inorganic acids) such as hydrochloric acid, nitric acid, sulfuric acid, phosphoric acid, or combinations thereof; and any combination thereof. In some embodiments, the acid is citric acid.

In some embodiments, the amount of acid is 0.001 wt-% or greater, 0.01 wt-% or greater, 0.1 wt-% or greater, 0.2 wt-% or greater, 0.5 wt-% or greater, or 1 wt-% or greater of the total formulation. In some embodiments, the amount of acid is 5 wt-% or less, 1 wt-% or less, 0.5 wt-% or less, 0.2 wt-% or less, 0.1 wt-% or less, or 0.01 wt-% or less of the total formulation. In some embodiments, the amount of acid is 0.001 wt-% to 5 wt-%, 0.001 wt-% to 1 wt-%, 0.001 wt-% to 0.5 wt-%, 0.001 wt-% to 0.2 wt-%, 0.001 wt-% to 0.1 wt-%, or 0.001 wt-% to 0.01 wt-% of the total formulation. In some embodiments, the amount of acid is 0.01 wt-% to 5 wt-%, 0.01 wt-% to 1 wt-%, 0.01 wt-% to 0.5 wt-%, 0.01 wt-% to 0.2 wt-%, or 0.01 wt-% to 0.1 wt-% of the total formulation. In some embodiments, the amount of acid is 0.1 wt-% to 5 wt-%, 0.1 wt-% to 1 wt-%, 0.1 wt-% to 0.5 wt-%, or 0.1 wt-% to 0.2 wt-% of the total formulation. In some embodiments, the amount of acid is 0.2 wt-% to 5 wt-%, 0.2 wt-% to 1 wt-%, or 0.2 wt-% to 0.5 wt-% of the total formulation. In some embodiments, the amount of acid is 0.5 wt-% to 5 wt-% or 0.5 wt-% to 1 wt-% of the total formulation. In some embodiments, the amount of acid is 1 wt-% to 5 wt-% of the total formulation.

In some embodiments, the amount of citric acid is 0.001 wt-% or greater, 0.004 wt-% or greater, 0.01 wt-% or greater, 0.1 wt-% or greater, 0.2 wt-% or greater, 0.5 wt-% or greater, or 1 wt-% or greater of the total formulation. In some embodiments, the amount of citric acid is 5 wt-% or less, 1 wt-% or less, 0.5 wt-% or less, 0.4 wt-% or less, 0.2 wt-% or less, 0.1 wt-% or less, or 0.01 wt-% or less of the total formulation. In some embodiments, the amount of citric acid is 0.001 wt-% to 5 wt-%, 0.001 wt-% to 1 wt-%, 0.001 wt-% to 0.5 wt-%, 0.001 wt-% to 0.2 wt-%, 0.001 wt-% to 0.1 wt-%, or 0.001 wt-% to 0.01 wt-% of the total formulation. In some embodiments, the amount of citric acid is 0.01 wt-% to 5 wt-%, 0.01 wt-% to 1 wt-%, 0.01 wt-% to 0.5 wt-%, 0.01 wt-% to 0.2 wt-%, or 0.01 wt-% to 0.1 wt-% of the total formulation. In some embodiments, the amount of citric acid is 0.1 wt-% to 5 wt-%, 0.1 wt-% to 1 wt-%, 0.1 wt-% to 0.5 wt-%, or 0.1 wt-% to 0.2 wt-% of the total formulation. In some embodiments, the amount of citric acid is 0.2 wt-% to 5 wt-%, 0.2 wt-% to 1 wt-%, or 0.2 wt-% to 0.5 wt-% of the total formulation. In some embodiments, the amount of citric acid is 0.5 wt-% to 5 wt-% or 0.5 wt-% to 1 wt-% of the total formulation. In some embodiments, the amount of citric acid is 1 wt-% to 5 wt-% of the total formulation. In some embodiments, the amount of citric acid is 0.004 wt-% to 0.4 wt-% of the total formulation.

In some embodiments, the formulation includes a co-solvent. In some embodiments the co-solvent is an alcohol, such as ethanol.

In some embodiments, the amount of the co-solvent is 0.5 wt-% or greater, 5 wt-% or greater, 10 wt-% or greater, 15 wt-% or greater, 17.5 wt-% or greater, or 20 wt-% or greater of the total composition. In some embodiments, the amount of the co-solvent is 25 wt-% or less, 20 wt-% or less, 17.5 wt-% or less, 15 wt-% or less, 10 wt-% or less, or 5 wt-% or less. In some embodiments, the amount of the co-solvent is 0.5 wt-% to 25 wt-%, 0.5 wt-% to 20 wt-%, 0.5 wt-% to 15 wt-%, 0.5 wt-% to 10 wt-% or 0.5 wt-% to 5 wt-% of the total formulation. In some embodiments, the amount of the co-solvent is 5 wt-% to 25 wt-%, 5 wt-% to 20 wt-%, 5 wt-% to 15 wt-%, or 5 wt-% of the total formulation. In some embodiments, the amount of the co-solvent is 10 wt-% to 25 wt-%, 10 wt-% to 20 wt-%, or 10 wt-% to 15 wt-% of the total formulation. In some embodiments, the amount of the co-solvent is 15 wt-% to 25 wt-% or 15 wt-% to 20 wt-% of the total formulation. In some embodiments, the amount of the co-solvent is 20 wt-% to 25 wt-% of the total formulation.

In some embodiments, the co-solvent is ethanol and the amount of ethanol is 0.5 wt-% or greater, 5 wt-% or greater, 10 wt-% or greater, 15 wt-% or greater, 17.5 wt-% or greater, or 20 wt-% or greater of the total composition. In some embodiments, the amount of ethanol is 25 wt-% or less, 20 wt-% or less, 17.5 wt-% or less, 15 wt-% or less, 10 wt-% or less, or 5 wt-% or less. In some embodiments, the amount of ethanol is 0.5 wt-% to 25 wt-%, 0.5 wt-% to 20 wt-%, 0.5 wt-% to 15 wt-%, 0.5 wt-% to 10 wt-% or 0.5 wt-% to 5 wt-% of the total formulation. In some embodiments, the amount of the ethanol is 5 wt-% to 25 wt-%, 5 wt-% to 20 wt-%, 5 wt-% to 15 wt-%, or 5 wt-% of the total formulation. In some embodiments, the amount of the ethanol is 10 wt-% to 25 wt-%, 10 wt-% to 20 wt-%, or 10 wt-% to 15 wt-% of the total formulation. In some embodiments, the amount of ethanol is 15 wt-% to 25 wt-% or 15 wt-% to 20 wt-% of the total formulation. In some embodiments, the amount of the ethanol is 20 wt-% to 25 wt-% of the total formulation.

In some embodiments, the composition includes 0.02 wt-% to 0.08 wt-% of ipratropium, ipratropium bromide, ipratropium bromide monohydrate, and/or anhydrous ipratropium bromide; 70 wt-% to 99.9 wt-% IFO-1234ze(E) or IFA-152a; 0.1 wt-% to 10 wt-% of water; 0.001 wt-% to 5 wt-% citric acid; and 0.5 wt-% to 25 wt-% ethanol. In some embodiments, the composition includes 0.02 wt-% to 0.06 wt-% of ipratropium, ipratropium bromide, and/or anhydrous ipratropium bromide; 75 wt-% to 90 wt-% HFO-1234ze(E) or HFA-152a; 0.1 wt-% to 1 wt-% of water; 0.001 wt-% to 1 wt-% citric acid; and 5 wt-% to 20 wt-% ethanol.

In some embodiments, the composition includes 0.02 wt-% to 0.06 wt-% of ipratropium, ipratropium bromide, and/or anhydrous ipratropium bromide; 80 wt-% to 90 wt-% HFO-1234ze(E) or HFA-152a; 0.1 wt-% to 0.5 wt-% of water; 0.01 wt-% to 0.5 wt-% citric acid; and 5 wt-% to 15 wt-% ethanol.

In some embodiments, the composition includes 0.002 wt-% to 0.08 wt-% of tiotropium, tiotropium bromide, tiotropium bromide monohydrate, and/or anhydrous tiotropium bromide; 70 wt-% to 99.9 wt-% HFO-1234ze(E) or HFA-152a; 0.001 wt-% to 5 wt-% citric acid; and 0.5 wt-% to 25 wt-% ethanol.

In some embodiments, the composition includes 0.01 wt-% to 0.05 wt-% of tiotropium, tiotropium bromide, and/or anhydrous tiotropium bromide; 75 wt-% to 90 wt-% HFO-1234ze(E) or HFA-152a; 0.001 wt-% to 1 wt-% citric acid; and 5 wt-% to 25 wt-% ethanol.

In some embodiments, the composition includes 0.01 wt-% to 0.04 wt-% of tiotropium, tiotropium bromide, and/or anhydrous tiotropium bromide; 75 wt-% to 90 wt-% HFO-1234ze(E) or HFA-152a; 0.01 wt-% to 0.5 wt-% citric acid; and 15 wt-% to 22.5 wt-% ethanol.

In some embodiments, the composition includes 0.005 wt-% to 0.1 wt-% of glycopyrronium and/or glycopyrronium bromide; 70 wt-% to 99.9 wt-% HFO-1234ze(E) or HFA-152a; 0.001 wt-% to 0.1 wt-% hydrochloric acid; and 5 wt-% to 25 wt-% ethanol.

In some embodiments, the composition includes 0.01 wt-% to 0.08 wt-% of glycopyrronium and/or glycopyrronium bromide; 75 wt-% to 90 wt-% HFO-1234ze(E) or HFA-152a; 0.015 wt-% to 0.06 wt-% hydrochloric acid; and 10 wt-% to 25 wt-% ethanol.

In some embodiments, the composition includes 0.01 wt-% to 0.05 wt-% of glycopyrronium and/or glycopyrronium bromide; 75 wt-% to 90 wt-% HFO-1234ze(E) or HFA-152a; 0.02 wt-% to 0.04 wt-% hydrochloric acid; and 10 wt-% to 20 wt-% ethanol.

The total amount of composition is desirably selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. The total amount of composition in the canister may be from 1.0 grams (g) to 30.0 g, 2.0 g to 20.0 g, 5.0 to 10.0 g. The total amount of composition is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times, the product of the predetermined number of doses and the metering volume of the metering valve. This ensures that the amount of each dose remains relatively constant through the life of the inhaler.

In some embodiments, additional components beyond propellant and API can be added to the formulation. These components may have various uses and functions, including, but not limited to, aiding in dissolution of API or other components, and/or aiding in chemical stabilization of API or other components.

In some embodiments a cosolvent is employed. One particularly useful cosolvent is ethanol. When used, the cosolvent, most particularly ethanol, may be in amounts on a weight percent basis of the total formulation of between 0.5% and 25%, between 1% and 22.5%, between 2% and 22.5%, between 5% and 22.5%, or between 10% and 22.5%.

In some embodiments, ethanol is used as a cosolvent in solution formulations, i.e., where the API is dissolved in the formulation. In one aspect, the ethanol may aid in dissolving the API whereas the API may not be soluble in the formulation in the absence of ethanol. When used in solution formulations, ethanol may be in amounts on a weight percent basis of the total formulation of between 2% and 25%, between 5% and 22.5%, or between 10% and 22.5%.

In certain embodiments, compositions of the present disclosure preferably display physical stability such that no particles are visible for at least 18 months, and often from 24 to 36 months under typical storage conditions In certain embodiments, compositions of the present disclosure preferably display chemical stability such that acceptable levels of degradation products are present in the finished product for at least 18 months, and often from 24 to 36 months under typical storage conditions.

Returning to FIG. 1, in use, the patient actuates the inhaler 100 by pressing downwardly on the canister 1. This moves the canister 1 into the body of the actuator 5 and presses the valve stem 14 against the actuator stem socket resulting in the canister metering valve opening and releasing a metered dose of composition that passes through the actuator nozzle 7 and exits the mouthpiece 6 into the patient's mouth. It should be understood that other modes of actuation, such as breath-actuation, may be used as well and would operate as described with the exception that the force to depress the canister would be provided by the device, for instance by a spring or a motor-driven screw, in response to a triggering event, such as patient inhalation.

Devices that may be used with medicament compositions of the present disclosure include those described in U.S. Pat. No. 6,032,836 (Hiscocks et al.), U.S. Pat. No. 9,010,329 (Hansen), and U.K. Patent GB 2544128 B (Friel).

The metered dose inhaler can include a dose counter for counting the number of doses. Suitable dose counters are known in the art, and are described in, for example, U.S. Pat. No. 8,740,014 (Purkins et al.); U.S. Pat. No. 8,479,732 (Stuart et al.); and U.S. Pat. No. 8,814,035 (Stuart), and U.S. Patent Application Publication No. 2012/0234317 (Stuart), all of which are incorporated by reference in their entirety with respect to their disclosures of dose counters.

One exemplary dose counter, which is described in detail in U.S. Pat. No. 8,740,014 (Purkins et al., hereby incorporated by reference in its entirety for its disclosure of the dose counter) has a fixed ratchet element and a trigger element that is constructed and arranged to undergo reciprocal movement coordinated with the reciprocal movement between an actuation element in an inhaler and the dose counter. The reciprocal movement can include an outward stroke (outward being with respect to the inhaler) and a return stroke. The return stroke returns the trigger element to the position that it was in prior to the outward stroke. A counter element is also included in this type of dose counter. The counter element is constructed and arranged to undergo a predetermined counting movement each time a dose is dispensed. The counter element is biased towards the fixed ratchet and trigger elements and is capable of counting motion in a direction that is substantially orthogonal to the direction of the reciprocal movement of the trigger element.

The counter element in the above-described dose counter includes a first region for interacting with the trigger member. The first region includes at least one inclined surface that is engaged by the trigger member during the outward stroke of the trigger member.

This engagement during the outward stroke causes the counter element to undergo a counting motion. The counter element also includes a second region for interacting with the ratchet member. The second region includes at least one inclined surface that is engaged by the ratchet element during the return stroke of the trigger element causing the counter element to undergo a further counting motion, thereby completing a counting movement. The counter element is normally in the form of a counter ring, and is advanced partially on the outward stroke of the trigger element, and partially on the return stroke of the trigger element. As the outward stroke of the trigger can correspond to the depression of a valve stem that causes firing of the valve (and, in the case of a metered dose inhaler, also meters the contents) and the return stroke can correspond to the return of the valve stem to its resting position, this dose counter allows for precise counting of doses.

Another suitable dose counter, which is described in detail in U.S. Pat. No. 8,479,732 (Stuart et al., hereby incorporated by reference in its entirety for its disclosure of dose counters) is specially adapted for use with a metered dose inhaler. This dose counter includes a first count indicator having a first indicia bearing surface. The first count indicator is rotatable about a first axis. The dose counter also includes a second count indicator having a second indicia bearing surface. The second count indicator is rotatable about a second axis. The first and second axes are disposed such that they form an obtuse angle. The obtuse angle mentioned above can be any obtuse angle, but is advantageously 125 to 145 degrees. The obtuse angle permits the first and second indicia bearing surface to align at a common viewing area to collectively present at least a portion of a medication dosage count. One or both of the first and second indicia bearing surfaces can be marked with digits, such that when viewed together through the viewing area the numbers provide a dose count. For example, one of the first and second indicia bearing surface may have “hundreds” and “tens” place digits, and the other with “ones” place digits, such that when read together the two indicia bearing surfaces provide a number between 000 and 999 that represents the dose count.

Yet another suitable dose counter is described in U.S. Patent Application Publication No. 2012/0234317 (Stuart, hereby incorporated by reference in its entirety for its disclosure of dose counters). Such a dose counter includes a counter element that undergoes a predetermined counting motion each time a dose is dispensed. The counting motion can be vertical or essentially vertical. A count indicating element is also included.

The count indicating element, which undergoes a predetermined count indicating motion each time a dose is dispensed, includes a first region that interacts with the counter element.

The counter element has regions for interacting with the count indicating element. Specifically, the counter element includes a first region that interacts with a count indicating element. The first region includes at least one surface that it engaged with at least one surface of the first region of the aforementioned count indicating element. The first region of the counter element and the first surface of the count inducing element are disposed such that the count indicating member completes a count indicating motion in coordination with the counting motion of the counter element, during and induced by the movement of the counter element, the count inducing element undergoes a rotational or essentially rotational movement. In practice, the first region of the counter element or the counter indicating element can include, for example, one or more channels. A first region of the other element can include one or more protrusions adapted to engage with said one or more channels.

Yet another dose counter is described in U.S. Pat. No. 8,814,035 (Stuart, hereby incorporated by reference in its entirety for its disclosure of dose counters). Such a dose counter is specially adapted for use with an inhaler with a reciprocal actuator operating along a first axis. The dose counter includes an indicator element that is rotatable about a second axis. The indicator element is adapted to undergo one or more predetermined count-indicating motions when one or more doses are dispensed. The second axis is at an obtuse angle with respect to the first axis. The dose counter also contains a worm rotatable about a worm axis. The worm is adapted to drive the indicator element. It may do this, for example, by containing a region that interacts with and enmeshes with a region of the indicator element. The worm axis and the second axis do not intersect and are not aligned in a perpendicular manner. The worm axis is also, in most cases, not disposed in coaxial alignment with the first axis. However, the first and second axes may intersect.

At least one of the various internal components of an inhaler, such as a metered dose inhaler, as described herein, such as one or more of the canister, valve, gaskets, seals, or O-rings, can be coated with one or more coatings. Some of these coatings provide a low surface energy. Such coatings are not required because they are not necessary for the successful operation of all inhalers.

Some coatings that can be used are described in U.S. Pat. No. 8,414,956 (Jinks et al.), U.S. Pat. No. 8,815,325 (David et al.), and U.S. Patent Application Publication No. 2012/0097159 (Iyer et al.), all of which are incorporated by reference in their entireties for their disclosure of coatings for inhalers and inhaler components. Other coatings, such as fluorinated ethylene propylene resins, or FEP, are also suitable. FEP is particularly suitable for use in coating canisters.

A first acceptable coating can be provided by the following method:
a) providing one or more component of the inhaler, such as the metered dose inhaler,
b) providing a primer composition including a silane having two or more reactive silane groups separated by an organic linker group,
c) providing a coating composition including an at least partially fluorinated compound,
d) applying the primer composition to at least a portion of the surface of the component,
e) applying the coating composition to the portion of the surface of the component after application of the primer composition.

The at least partially fluorinated compound will usually include one or more reactive functional groups, with at least one reactive functional group usually being a reactive silane group, for example a hydrolysable silane group or a hydroxysilane group. Such reactive silane groups allow reaction of the partially fluorinated compound with one or more of the reactive silane groups of the primer. Often such reaction will be a condensation reaction.

One exemplary silane that can be used has the formula

X₃₋ₘ(R¹)ₘSi-Q-Si(R²)ₖX₃₋ₖ
wherein R¹ and R² are independently selected univalent groups, X is a hydrolysable or hydroxy group, m and k are independently 0, 1, or 2 and Q is a divalent organic linking group.

Useful examples of such silanes include one or a mixture of two or more of 1,2-bis(trialkoxysilyl) ethane, 1,6-bis(trialkoxysilyl) hexane, 1,8-bis(trialkoxysilyl) octane, 1,4-bis(trialkoxysilylethyl)benzene, bis(trialkoxysilyl)itaconate, and 4,4′-bis(trialkoxysilyl)-1,1′-diphenyl, wherein any trialkoxy group may be independently trimethoxy or triethoxy.

The coating solvent usually includes an alcohol or a hydrofluoroether.

If the coating solvent is an alcohol, preferred alcohols are C₁ to C₄ alcohols, in particular, an alcohol selected from ethanol, n-propanol, or isopropanol or a mixture of two or more of these alcohols.

If the coating solvent is an hydrofluoroether, it is preferred if the coating solvent includes a C₄ to C₁₀ hydrofluoroether. Generally, the hydrofluoroether will be of formula

C_{g}F_{2g+1}OCₕH₂ₕ₊₁
wherein g is 2, 3, 4, 5, or 6 and h is 1, 2, 3 or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

The polyfluoropolyether silane can be of the formula

R^{f}Q¹ᵥ[Q²_{w}-[C(R⁴)₂—Si(X)₃₋ₓ(R⁵)ₓ]_{y}]_{z}
wherein:
   R^{f} is a polyfluoropolyether moiety;
   Q¹ is a trivalent linking group;
   each Q² is an independently selected organic divalent or trivalent linking group;
each R⁴ is independently hydrogen or a C₁₋₄ alkyl group;
each X is independently a hydrolysable or hydroxyl group;
R⁵ is a C₁₋₈ alkyl or phenyl group;
v and w are independently 0 or 1, x is 0 or 1 or 2; y is 1 or 2; and z is 2, 3, or 4.

The polyfluoropolyether moiety R^{f} can include perfluorinated repeating units selected from the group consisting of —(CₙF₂ₙO)—, —(CF(Z)O)—, —(CF(Z)CₙF₂O)—, —(CₙF₂ₙCF(Z)O)—, —(CF₂CF(Z)O)—, and combinations thereof, wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted and wherein for repeating units including Z the number of carbon atoms in sequence is at most 6. In particular, n can be an integer from 1 to 4, more particularly from 1 to 3. For repeating units including Z the number of carbon atoms in sequence may be at most four, more particularly at most 3. Usually, n is 1 or 2 and Z is an —CF₃ group, more wherein z is 2, and R^{f} is selected from the group consisting of —CF₂O(CF₂O)ₘ(C₂F₄O)ₚCF₂—, —CF(CF₃)O(CF(CF₃)CF₂O)ₚCF(CF₃)—, —CF₂O(C₂F₄O)ₚCF₂—, —(CF₂)₃O(C₄F₈O)ₚ(CF₂)₃—, —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

A cross-linking agent can be included. Exemplary cross-linking agents include tetramethoxysilane; tetraethoxysilane; tetrapropoxysilane; tetrabutoxysilane; methyl triethoxysilane; dimethyldiethoxysilane; octadecyltriethoxysilane; 3-glycidoxy-propyltrimethoxysilane; 3-glycidoxy-propyltriethoxysilane; 3-aminopropyl-trimethoxysilane; 3-aminopropyl-triethoxysilane; bis(3-trimethoxysilylpropyl) amine; 3-aminopropyl tri(methoxyethoxyethoxy) silane; N-(2-aminoethyl)3-aminopropyltrimethoxysilane; bis(3-trimethoxysilylpropyl) ethylenediamine; 3-mercaptopropyltrimethoxysilane; 3-mercaptopropyltriethoxysilane; 3-trimethoxysilyl-propylmethacrylate; 3-triethoxysilypropylmethacrylate; bis(trimethoxysilyl) itaconate; allyltriethoxysilane; allyltrimethoxysilane; 3-(N-allylamino)propyltrimethoxysilane; vinyltrimethoxysilane; vinyltriethoxysilane; and mixtures thereof.

The component to be coated can be pre-treated before coating, such as by cleaning. Cleaning can be by way of a solvent, such as a hydrofluoroether, e.g., HFE72DE, or an azeotropic mixture of 70% w/w (weight percent) trans-dichloroethylene; 30% w/w of a mixture of methyl and ethyl nonafluorobutyl and nonafluoroisobutyl ethers.

The above-described first acceptable coating is particularly useful for coating valves components, including one or more of valve stems, bottle emptiers, springs, and tanks. This coating system can be used with any type of inhaler and any formulation described herein.

In some embodiments the actuator nozzle is sized so as to optimize the fine particle fraction (FPF) and/or respirable dose delivered of the formulation within the canister. In some embodiments the cross-sectional shape of the actuator nozzle is essentially circular or circular and has a predetermined diameter. In some embodiments where the cross-sectional shape of the actuator nozzle is non-circular, for example oval, an effective diameter may be determined by taking an average over the distances spanning the opening (e.g., the average of major and minor axes of an ellipse).

In some embodiments the exit orifice (effective diameter) of the actuator nozzle may be 0.08 mm or greater, 0.10 or greater, 0.12 mm or greater, 0.15 mm or greater, 0.175 mm or greater, 0.225 mm or greater 0.3 mm or greater, or 0.4 mm or greater. In some embodiments the exit orifice (effective diameter) of the actuator nozzle may be 0.5 mm or less, 0.4 mm or less, 0.3 mm or less, 0.225 mm or less, 0.175 mm or less, or 0.15 mm or less. In some embodiments the exit orifice (effective diameter) of the actuator nozzle may be 0.12 mm to 0.5 mm, 0.12 mm to 0.4 mm, 0.12 mm to 0.3 mm, 0.12 mm to 0.225 mm, 0.12 mm to 0.175 mm, or 0.12 mm to 0.15 mm. In some embodiments the exit orifice (effective diameter) of the actuator nozzle may be 0.15 mm to 0.5 mm, 0.15 mm to 0.4 mm, 0.15 mm to 0.3 mm, 0.15 mm to 0.225 mm, or 0.15 mm to 0.175 mm. In some embodiments the exit orifice (effective diameter) of the actuator nozzle may be 0.175 mm to 0.5 mm, 0.175 mm to 0.4 mm, 0.175 mm to 0.3 mm, or 0.175 mm to 0.225 mm. In some embodiments the exit orifice (effective diameter) of the actuator nozzle may be 0.12 mm to 0.5 mm, 0.14 mm to 0.4 mm, or 0.18 mm to 0.3 mm. In some embodiments the exit orifice (effective diameter) of the actuator nozzle may be 0.12 mm to 0.3 mm or 0.18 mm to 0.22 mm. In some embodiments the exit orifice (effective diameter) of the actuator nozzle may be 0.12 mm to 0.25 mm.

In some embodiments, the MDI is manufactured by pressure filling. In pressure filling, the liquid or powdered medicament, combined with one or more excipients (e.g., co-solvents), is placed in a suitable aerosol container (i.e., canister) capable of withstanding the vapor pressure of the propellant and fitted with a metering valve prior to filling. The propellant is then forced as a liquid through the valve into the container. In an alternate process of pressure filling, the particulate drug is combined in a process vessel with propellant and one or more excipients (e.g., cosolvents), and the resulting drug solution is transferred through the metering valve fitted to a suitable MDI container.

In some embodiments, the MDI is manufactured by cold filling. In cold filling, the liquid or powdered medicament is combined with one or more excipients (e.g., co-solvents) and propellant which is chilled below its boiling point and, optionally, one or more excipients are added to the MDI container. In addition, a metering valve is fitted to the container post filling.

For both pressure filling and cold filling processes, additional steps, such as mixing, sonication, and homogenization may be optionally employed.

### Embodiments

Embodiment 1 is a composition comprising a solution comprising: an active pharmaceutical ingredient comprising (preferably, consists essentially of, and more preferably, consists of) an anticholinergic agent; and a propellant comprising HFA-152a, HFO1234ze(E), or both.

Embodiment 2 is the composition of embodiment 1, wherein the anticholinergic agent comprises a long-acting muscarinic antagonist.

Embodiment 3 is the composition of embodiment 1 or 2, wherein the anticholinergic agent is selected from ipratropium, tiotropium, aclidinium, umeclidinium, glycopyrronium, a pharmaceutically acceptable salt or ester of any of the listed drugs, or a mixture of any of the listed drugs, their pharmaceutically acceptable salts or their pharmaceutically acceptable esters.

Embodiment 4 is the composition of embodiment 1 or 2, wherein the anticholinergic agent comprises a quaternary ammonium salt.

Embodiment 5 is the composition of any preceding embodiment, wherein the anticholinergic agent is selected from ipratropium, tiotropium, glycopyrronium, and a pharmaceutically acceptable salt or ester of any of the listed drugs.

Embodiment 6 is the composition of any preceding embodiment, wherein the anticholinergic agent is selected from ipratropium bromide, tiotropium bromide, and glycopyrronium bromide.

Embodiment 7 is the composition of any preceding embodiment, wherein the sole anticholinergic agent is tiotropium bromide.

Embodiment 8 is the composition of any preceding embodiment, wherein the sole anticholinergic agent is glycopyrronium bromide.

Embodiment 9 is a composition comprising a solution comprising: ipratropium bromide; and HFA-152a, HFO1234ze(E), or both.

Embodiment 10 is a composition comprising a solution comprising: ipratropium bromide and HFO1234ze(E).

Embodiment 11 is a composition comprising a solution comprising: ipratropium bromide and HFA-152a.

Embodiment 12 is a composition comprising a solution comprising: an active pharmaceutical ingredient consisting essentially of (preferably consisting of) ipratropium bromide; and a propellant comprising HFA-152a, HFO-1234ze(E), or both.

Embodiment 13 is the composition of any preceding embodiment, the solution further comprising ethanol.

Embodiment 14 is the composition of embodiment 13, wherein ethanol is present at 5 wt-% to 25 wt-% of the total composition.

Embodiment 15 is the composition of embodiment 14, wherein ethanol is present at 10 wt-% to 22.5 wt-% of the total composition.

Embodiment 16 is the composition of any preceding embodiment, the solution further comprising water.

Embodiment 17 is the composition of embodiment 16, wherein the water is present at 0.1 wt-% to 1 wt-% of the total composition Embodiment 18 is the composition of embodiment 17, wherein the water is present at 0.4 wt-% to 0.6 wt-%.

Embodiment 19 is the composition of any preceding embodiment, the solution further comprising an acid.

Embodiment 20 is the composition of embodiment 19, wherein the acid comprises an organic acid, inorganic acid, or a combination thereof.

Embodiment 21 is the composition of embodiment 20, wherein the acid is an inorganic acid.

Embodiment 22 is the composition of embodiment 21, wherein the inorganic acid is selected from hydrochloric acid, nitric acid, phosphoric acid, sulfuric acid, and a combination thereof.

Embodiment 23 is the composition of embodiment 22, wherein the acid is hydrochloric acid.

Embodiment 24 is the composition of embodiment 20, wherein the acid is an organic acid.

Embodiment 25 is the composition of embodiment 24, wherein the organic acid is selected from citric acid, ascorbic acid, maleic acid, acetic acid, succinic acid, formic acid, fumaric acid, propionic acid, oxalic acid, lactic acid, glycolic acid, and a combination thereof.

Embodiment 26 is the composition of embodiment 25, wherein the acid is citric acid.

Embodiment 27 is the composition of any of embodiments 20 to 26, wherein the acid is present at a concentration of 0.001 wt-% to 5 wt-% of the total composition.

Embodiment 28 is the composition of embodiment 27, wherein the acid is present at 0.01 wt-% to 0.5 wt-% of the total composition.

Embodiment 29 is the composition of any of embodiments 16 to 28, wherein water is present at 0.1 wt % to 1 wt-% of the total composition.

Embodiment 30 is the composition of embodiment 29, wherein water is present at 0.4 wt-% to 0.6 wt-% of the total composition.

Embodiment 31 is the composition of any preceding embodiment, wherein HFA152a, HFO1234ze(E), or a combination thereof makes up 75 wt-% to 95 wt-% of the total composition.

Embodiment 32 is the composition of embodiment 31, wherein HFA152a, HFO1234ze(E), or a combination thereof makes up 75 wt-% to 90 wt-% of the total composition.

Embodiment 33 is the composition of any preceding embodiment, wherein the API (particularly ipratropium bromide) is present at 0.01 wt-% to 0.08 wt-% of the total composition.

Embodiment 34 is the composition of embodiment 33, wherein API (particularly ipratropium bromide) is present at 0.02 wt-% to 0.06 wt-% of the total composition.

Embodiment 35 is a composition comprising a solution comprising: ipratropium bromide; HFA152a, HFO1234ze(E), or both; an acid; water; and ethanol.

Embodiment 36 is a composition comprising a solution comprising: ipratropium bromide; HFO1234ze(E), or both; an acid; water; and ethanol.

Embodiment 37 is a composition comprising a solution comprising: ipratropium bromide; HFA152a; an acid; water; and ethanol.

Embodiment 38 is the composition of any of embodiments 35 to 37, wherein the solution exhibits improved physical stability when stored at 40° C. and 70% relative humidity for at least 6 weeks as compared to a formulation without ethanol or citric acid.

Embodiment 39 is the composition of any of embodiments 35 to 38, wherein the solution exhibits improved physical stability when stored at 40° C. and 70% relative humidity for at least 13 weeks as compared to a formulation without ethanol or citric acid.

Embodiment 40 is the composition of any of embodiments 35 to 39, wherein the solution exhibits improved physical stability when stored at 40° C. and 70% relative humidity for at least 12 months as compared to a formulation without ethanol or citric acid.

Embodiment 41 is the composition of any of embodiments 35 to 40, wherein the solution exhibits lower levels of chemical impurities as compared to a solution without acid or ethanol.

Embodiment 42 is a composition comprising a solution comprising: tiotropium bromide; HFA152a, HFO1234ze(E), or both; an acid; and ethanol.

Embodiment 43 is a composition comprising a solution comprising: tiotropium bromide; HFO1234ze(E); an acid; and ethanol.

Embodiment 44 is a composition comprising a solution comprising: tiotropium bromide; HFA152a; an acid; and ethanol.

Embodiment 45 is the composition of any of embodiments 42 to 44, wherein the ethanol is present at a concentration of at least 17.5 wt-%.

Embodiment 46 is the composition of any of embodiments 42 to 44, wherein the solution further comprises acid.

Embodiment 47 is the composition of embodiment 46, wherein the acid is citric acid.

Embodiment 48 is the composition of embodiment 47, wherein the citric acid is present at a concentration of at least 0.04.

Embodiment 49 is the composition of any preceding embodiment, wherein the API (particularly tiotropium bromide) is present at 0.005 wt-% to 0.1 wt-% of the total composition.

Embodiment 50 is the composition of any preceding embodiment, wherein API (particularly tiotropium bromide) is present at 0.01 wt-% to 0.04 wt-% of the total composition.

Embodiment 51 is the composition of any of embodiments 42 to 50, wherein the solution exhibits improved physical stability when stored at 40° C. and 70% relative humidity for at least 6 weeks as compared to a formulation without ethanol or citric acid.

Embodiment 52 is the composition of any of embodiments 42 to 51, wherein the solution exhibits improved physical stability when stored at 40° C. and 70% relative humidity for at least 13 weeks as compared to a formulation without ethanol or citric acid.

Embodiment 53 is the composition of any of embodiments 42 to 52, wherein the solution exhibits improved physical stability when stored at 40° C. and 70% relative humidity for at least 12 months as compared to a formulation without ethanol or citric acid.

Embodiment 54 is the composition of any of embodiments 42 to 53, wherein the solution exhibits lower levels of chemical impurities as compared to a solution without acid or ethanol.

Embodiment 55 is a composition comprising a solution comprising: glycopyrronium bromide; HFA152a, HFO1234ze(E), or both; an acid; and ethanol.

Embodiment 56 is a composition comprising a solution comprising: glycopyrronium bromide; HFO1234ze(E); an acid; and ethanol.

Embodiment 57 is a composition comprising a solution comprising: glycopyrronium bromide; HFA152a; an acid; and ethanol.

Embodiment 58 is the composition of any of embodiments 55 to 57, wherein the ethanol is present at a concentration of at least 17.5 wt-%.

Embodiment 59 is the composition of any of embodiments 55 to 58, wherein the solution further comprises acid.

Embodiment 60 is the composition of embodiment 59, wherein the acid is hydrochloric acid.

Embodiment 61 is the composition of embodiment 60, wherein the hydrochloric acid is present at a concentration of at least 0.025%.

Embodiment 62 is the composition of any preceding embodiment, wherein the API (particularly glycopyrronium bromide) is present at 0.002 wt-% to 0.2 wt-% of the total composition.

Embodiment 63 is the composition of any preceding embodiment, wherein the API (particularly glycopyrronium bromide) is present at 0.005 wt-% to 0.1 wt-% of the total composition.

Embodiment 64 is the composition of any of embodiments 55 to 63, wherein the solution exhibits improved physical stability when stored at 40° C. and 70% relative humidity for at least 6 weeks as compared to a formulation without ethanol or hydrochloric acid.

Embodiment 65 is the composition of embodiment 64, wherein the solution exhibits improved physical stability when stored at 40° C. and 70% relative humidity for at least 13 weeks as compared to a formulation without ethanol or hydrochloric acid.

Embodiment 66 is the composition of embodiment 65, wherein the solution exhibits improved physical stability when stored at 40° C. and 70% relative humidity for at least 12 months as compared to a formulation without ethanol or hydrochloric acid.

Embodiment 67 is the composition of embodiment 66, wherein the solution exhibits lower levels of chemical impurities as compared to a solution without acid or ethanol.

Embodiment 68 is a metered dose inhaler comprising: a metering valve; a canister; and an actuator comprising an actuator nozzle; wherein the canister comprises the composition of any preceding embodiment.

Embodiment 69 is the metered dose inhaler of embodiment 68, wherein the metering valve comprises a metering chamber having a size between 25 microliters and 200 microliters.

Embodiment 70 is the metered dose inhaler of embodiment 69, wherein the metering chamber of the metering valve has a size between about 25 microliters to about 100 microliters.

Embodiment 71 is the metered dose inhaler of any of embodiments 68 to 70, wherein the actuator has an exit orifice diameter between 0.12 mm and 0.4 mm.

Embodiment 72 is the metered dose inhaler of embodiment 71, wherein the actuator exit orifice diameter is between 0.15 mm and 0.4 mm.

Embodiment 73 is the metered dose inhaler of embodiment 72, wherein the actuator exit orifice diameter is between 0.225 mm and 0.3 mm.

Embodiment 74 is the metered dose inhaler of any of embodiments 64 to 73, wherein the canister comprises from about 1 to about 30 mL of the composition.

Embodiment 75 is the metered dose inhaler of any of embodiments 64 to 74, wherein the canister contains a predetermined number of doses, and wherein the predetermined number of doses is from about 30 to about 200.

### EXAMPLES

### Comparative Example 1: Solubility of Tiotropium Bromide Monohydrate in HFA-134a, HFA-152a, and HFO-1234ze(E)

In this example, saturated solutions of tiotropium bromide (TBM) were prepared in three different propellants by adding excess drug to ensure saturated solubility was achieved. A first saturated solution including TB and the propellant HFO-1234ze(E) (1,3,3,3-tetrafluoropropene) was prepared. A second saturated solution including TB and the propellant HFA-134a (1,1,1,2-tetrafluoroethane) was prepared. A third saturated solution including TB and the propellant HFA-152a (1,1-difluoroethane) was prepared. For all three solutions, the concentration of TB used was 0.3750 mg/mL. The solutions did not contain any other added components.

A further set of saturated solutions was prepared using each propellant with the addition of 20% ethanol by weight and 0.4% citric acid by weight.

The TB solubility of all solutions was measured by a content assay test following filtration of the excess non-dissolved drug. It was observed that TB was practically insoluble in all propellants, although low levels of drug was detected in HFA-152a. Data from this example is shown in Table 1.

TABLE 1

| | Mean TB |
|---|---|
| Solution composition | solubility (mg/mL) |
| HFO-1234ze(E) | Below quantification |
| HFO-1234ze(E), | 0.0331 |
| 20% ethanol, | |
| 0.4% citric acid | |
| HFA-152a | 0.0005 |
| HFA-152a, | 0.2039 |
| 20% ethanol, | |
| 0.4% citric acid | |
| HFA-134a | Below quantification |
| HFA-134a, | 0.1220 |
| 20% ethanol, | |
| 0.4% citric acid | |

From this example, it was learned that TB was practically insoluble in all propellants, although low levels of drug was detected in HFA-152a. It was also learned that TB was notably more soluble in both HFA propellant solutions, compared to the HFO-1234ze(E) solution, with the addition of 20% ethanol and 0.4% citric acid by weight. This was most notable in HFA-152a. Therefore it is anticipated that physically stable solution compositions of TB in HFO-1234ze(E) will require higher levels of ethanol when compared to HFA compositions.

### Example 2: Solubility and Physical Stability of Tiotropium Bromide in Compositions Including HFO-1234ze(E) with Ethanol and Acid

In this example, solutions of TB in HFO-1234ze(E) including different amounts of ethanol and different acids were tested.

Solutions were prepared of 0.1204 mg/mL TBM in HFO-1234ze(E) with 10%, 12.5%, 15%, 17.5%, 20%, or 22.5% by weight of ethanol. An acid was added to each solution to test the interaction between TB, the given weight percentage of ethanol, and the acid. Six acids in total were tested in solutions across a range of ethanol concentrations for a total of 36 solutions total. Acids tested included citric acid, acetic acid, hydrochloric acid, succinic acid, ascorbic acid, and sulfuric acid. Solubility and physical stability of TB in each solution was visually inspected for up to 21 days at ambient conditions.

It was observed that all acids, other than citric acid, caused TB to precipitate. Solutions of 0.1250 mg/mL TB with citric acid concentrations between 0.04% and 0.4% by weight citric acid and each of the six tested weight percentages of ethanol were prepared and visually analyzed for seven days. It was observed that composition including less than 17.5% ethanol and each concentration of acid, TB precipitated from solution.

From this example, it was learned that formulations of TB in HFO-1234ze(E) with at least 17.5% by weight ethanol and with 0.04% by weight citric acid or greater were visually stable and remained in solution up to seven days post-preparation.

### Example 3: Chemical Stability of Tiotropium Bromide in Compositions Including in HFO-1234Ze(E), Ethanol, and Citric Acid

Three solutions including TB, ethanol, citric acid, and the propellant HFO-1234ze(E) were prepared. For all three solutions, the concentration of ethanol was 20% by weight and the concentration of TBM was 0.1250 mg/mL. The first solution included 0.04% by weight citric acid. The second solution included 0.22% by weight citric acid. The third solution included 0.4% by weight citric acid. Each solution was pressure filled into an FEP coated canister and fitted with a 50-μL BESPAK valve. The filled canisters were stored at 40° C. and 75% relative humidity for two weeks to simulate aging. Three replicates of each solution in total were prepared, packed, and stored.

After initial preparation and following two weeks' storage, each filled canister was analyzed for TB content and presence of impurities/degradants. Each solution was observed to have only a slight decrease in TB content after two weeks storage relative to the initial measured TB content as shown in Table 2. Each solution was also observed to have a low level of total impurities and known tiotropium degradants (with all impurities/degradants less than 0.2% by weight) as shown in Table 3.

TABLE 2

| | Citric Acid Level (% w/w) | | |
|---|---|---|---|
| Timepoint | 0.04% | 0.22% | 0.4% |
| Initial | 0.1026 | 0.1026 | 0.1012 |
| 2 weeks | 0.1009 | 0.1011 | 0.1001 |

TABLE 3

| | | Citric Acid Level (% w/w) | | |
|---|---|---|---|---|
| Timepoint | Impurity | 0.04% | 0.22% | 0.4% |
| Initial | Unknown 22 | 0.05 | 0.04 | 0.06 |
| | Unknown 21 | — | 0.04 | 0.03 |
| | Unknown 11 | 0.05 | 0.04 | 0.03 |
| | Total UV | 0.10 | 0.08 | 0.13 |
| | impurities | | | |
| 2 weeks | Ph. Eur. Impurity | 0.11 | 0.08 | 0.08 |
| | B | | | |
| | Ethyl Dithienyl | 0.05 | 0.04 | 0.06 |
| | Glycolate | | | |
| | Tiotropium Ethyl | — | — | 0.05 |
| | Ether | | | |
| | Total UV | 0.16 | 0.12 | 0.19 |
| | impurities | | | |

From this example, it was learned that compositions of TB in HFO-1234ze(E), 20% ethanol by weight and citric acid at levels between 0.04% to 0.4% citric acid by weight were relatively chemically stable over storage for two weeks at 40° C. and 75% relative humidity.

### Example 4: Comparison of Actuator Exit Orifice Sizes for Delivery of Tiotropium Bromide in Compositions of HFO-1234ze(E), Ethanol and Citric Acid

A solution composition including 0.125 mg/mL TBM, 0.22% by weight citric acid, and 20% by weight ethanol in HFO-1234ze(E) was prepared. The solution was pressure filled into FEP-coated canisters fitted with a 50-μL BESPAK Valve. Three units were prepared in total. One unit was tested with a KINDEVA Drug Delivery (KDD) actuator with a 0.3-mm exit orifice, one unit was tested with a KDD actuator with a 0.22 mm exit orifice, and one unit was tested with a KDD actuator with a 0.18 mm exit orifice

The fine particle mass (FPM) smaller than 5 m per actuation, median mass aerodynamic diameter (MMAD), delivered dose (ex-act), and geometric standard deviation (GSD) were measured for each unit tested. These measurements are shown in Table 4.

TABLE 4

| Exit orifice | FPM | MMAD | Total Ex-Act | |
|---|---|---|---|---|
| (mm) | (μg/act) | (μm) | (μg) | GSD |
| 0.18 | 2.4573 | 1.6537 | 4.6321 | 1.8323 |
| 0.22 | 1.5875 | 1.4980 | 4.7161 | 1.8477 |
| 0.30 | 0.9230 | 1.3393 | 4.5745 | 1.8817 |

From this example, it was observed that FPM is increased when the formulation was delivered using progressively smaller actuator exit orifices. It was learned that actuator exit orifices between 0.18 mm and 0.22 mm were preferred for delivery of anticipated appropriate FPM of a solution of TB in HFO-1234ze(E) with 20% ethanol by weight and 0.04% to 0.4% citric acid by weight.

### Comparative Example 5: Delivered Dose and APSD Measurement of Solutions of Ipratropium Bromide in HFA-152a, HFO-1234Ze(E) and HFA-134a

Three solution formulations of ipratropium bromide were prepared. Each solution formulation included 0.037% ipratropium bromide monohydrate by weight, 0.5% water by weight, 0.004% citric acid by weight, and 15% ethanol by weight. The first solution included HFA-152a. The second solution included HFO-1234ze(E). The third solution included HFA-134a. In all examples, a concentrate was made by combining acid, water, and ethanol, followed by API. The concentrates were sonicated to form a solution prior to the addition of propellant. All formulations were cold filled into FEP-coated cans and crimped with either a 50-μL BESPAK or a 50-μL APTAR valve.

Compositions were tested for KINDEVA through unit life delivered dose and aerodynamic particle size (APSD) (NGI and a USP induction port) using a KINDEVA actuator with a 0.25 mm exit orifice diameter. The mean results are presented in table 5 and table 6 below

TABLE 5
Mean through unit life delivered dose results

| | Start of | Middle of | End of | Overall |
|---|---|---|---|---|
| | unit life | unit life | unit life | unit |
| | delivered | delivered | delivered | delivered |
| | dose | dose | dose | dose |
| Configuration | (μg/act) | (μg/act) | (μg/act) | (μg/act) |
| HFA-152a- | 17.1 | 16.9 | 17.6 | 17.2 |
| BESPAK Valve | | | | |
| HFA-152a- | 16.9 | 16.4 | 17.1 | 16.8 |
| APTAR Valve | | | | |
| HFO-1234ze(E)- | 17.1 | 15.1 | 15.5 | 15.9 |
| BESPAK Valve | | | | |
| HFO-1234ze(E)- | 16.5 | 16.1 | 16.3 | 16.3 |
| APTAR Valve | | | | |
| HFA-134a- | 19.0 | 18.2 | 19.5 | 18.9 |
| BESPAK Valve | | | | |
| HFA-134a- | 18.2 | 17.8 | 18.1 | 18.0 |
| APTAR Valve | | | | |

TABLE 6
Mean APSD results

| | FPM | | | ISM | | | |
|---|---|---|---|---|---|---|---|
| | (>5 | | | (μg) | | Ex- | Ex- |
| | μm) | | | Cup | | Valve | Act |
| | (μg/ | MMAD | Throat | 1 to | FPF | (μg/ | (μg/ |
| Configuration | act) | (μm) | (μg) | filter | (%) | act) | act) |
| HFO-1234ze(E) − | 19 | 0.92 | 7.2 | 9.4 | 54.0 | 18.5 | 16.6 |
| APTAR Valve | | | | | | | |
| HFO-1234ze(E)/ | 9.3 | 0.92 | 7.0 | 9.7 | 56 | 18.7 | 16.7 |
| BESPAK Valve | | | | | | | |
| HFA-152a − | 9 | 0.93 | 7.6 | 9.5 | 53 | 19.4 | 17 |
| APTAR Valve | | | | | | | |
| HFA-152a − | 9.4 | 0.86 | 7.4 | 9.8 | 55 | 19.4 | 17.2 |
| BESPAK Valve | | | | | | | |
| HFA 134a − | 9.1 | 0.81 | 7.5 | 9.5 | 54 | 19.1 | 17.0 |
| APTAR Valve | | | | | | | |
| HFA 134a − | 9.1 | 0.80 | 8.1 | 9.5 | 52 | 20.2 | 17.6 |
| BESPAK Valve | | | | | | | |

It was observed that all compositions demonstrated the anticipated through unit life delivered dose and aerodynamic particle size (APSD) using both BESP and APTAR valves, and gave similar performance. Additionally, both compositions of HFO-1234ze (E) and HFA-152a gave similar through unit life delivered dose to the HFA-134a composition.

From this example, it was also learned that solutions of ipratropium bromide in HFA-152a or HFO-1234ze(E) with added acid and ethanol perform as anticipated and similarly with respect to through unit life delivered dose and aerodynamic particle size (APSD), and gave similar performance to that of HFA-134a compositions.

### Example 6: Solubility of Ipratropium Bromide in HFO-1234ze(E), HFA-152a, or HFA-134a with Increasing Concentrations of Ethanol

Saturated solutions of ipratropium bromide in HFO-1234ze(E), HFA-152a, or HFA-134a were prepared. Each solution contained 4 mg/mL of added ipratropium bromide, ensuring an excess of drug to saturate the solutions with respect to solubilized ipratropium bromide. A first set of solutions included only ipratropium bromide with each propellant. A second set of solutions included the addition of 1% ethanol by weight. A third set of solutions included the addition of 15% ethanol by weight, 0.004% citric acid by weight, and 0.5% water by weight. Each solution was pressure filled into containers. The containers were sonicated for 5-10 minutes, then shaken for 7-10 days. Each solution was then filtered to remove excess undissolved drug and the quantity of solubilized ipratropium bromide at saturation was determined. The results are shown in Table 6.

TABLE 6

| Ethanol | Ipratropium Bromide (mg/mL) | | |
|---|---|---|---|
| Concentration | Propellant | | |
| (by weight) | HFA-134a | HFA-152a | HFO-1234ze(E) |
| 0% | 0.002 | 0.002 | 0.000 |
| 1% | 0.005 | 0.004 | 0.000 |
| 15% | 1.397 | 1.367 | 0.210 |

It was observed that the saturated solubility of ipratropium bromide was significantly lower in HFO-1234ze (E) compositions with 15% ethanol, when compared to similar solutions in HFA-134a and HFA-152a

From this example, it was learned that ipratropium bromide was practically insoluble in all propellants, although low levels of drug was detected in both HFA propellants. It was also learned that ipratropium bromide was notably more soluble in both HFA propellant compositions, compared to the HFO-1234ze(E) compositions containing ethanol. This was notable in compositions with the addition of 15% ethanol and 0.004% citric acid by weight and 0.5% water by weight. Therefore it is anticipated that physically stable solution compositions of ipratropium bromide in HFO-1234ze(E) may require higher levels of ethanol to fully solubilize ipratropium bromide when compared to HFA-134a compositions. This is unlikely to be the case for ipratropium bromide HFA-152a compositions, when compared to HFA-134a compositions.

### Example 7: Stability of Solutions of Ipratropium Bromide in HFA-1234ze(E), HFA-152a, and HFA-134a

Solutions of ipratropium bromide monohydrate in HFA-134a, HFA-152a, and HFO-1234ze(E) were prepared. Each solution included ipratropium bromide monohydrate (0.4186 mg/mL), 15% ethanol by weight, 0.5% water by weight, and 0.004% citric acid by weight. Each solution was filled into FEP-coated aluminium, plain aluminium or stainless-steel canisters. Each canister was fitted with either a 50-μL BESPAK or a 50-μL APTAR valve.

Canisters were stored at 40° C. and 75% relative humidity for 13 weeks. At 13 weeks, and tested, in triplicate, for ipratropium bromide content, through unit life delivered dose (FEP coated canister compositions only) and impurities. Mean results are shown in Table 7, Table 8, and Table 9.

TABLE 7

| | | Ipratropium Content (mg/mL) | | |
|---|---|---|---|---|
| Composition | Valve/Canister | 0 Week | 6 Weeks | 13 Weeks |
| HFA-134a | BESPAK/Aluminium | 0.42 | 0.40 | 0.41 |
| | BESPAK/Stainless Steel | 0.55 | 0.45 | 0.44 |
| | BESPAK/FEP Coated | 0.42 | 0.40 | 0.40 |
| | APTAR/FEP Coated | 0.42 | 0.39 | 0.41 |
| HFA-152a | BESPAK/Aluminium | 0.42 | 0.41 | 0.41 |
| | BESPAK/Stainless Steel | 0.43 | 0.41 | 0.41 |
| | BESPAK/FEP Coated | 0.42 | 0.42 | 0.44 |
| | APTAR/FEP Coated | 0.42 | 0.42 | 0.41 |
| HFO-1234ze(E) | BESPAK/Aluminium | 0.43 | 0.42 | 0.41 |
| | BESPAK/Stainless Steel | 0.43 | 0.41 | 0.40 |
| | BESPAK/FEP Coated | 0.43 | 0.42 | 0.41 |
| | APTAR/FEP Coated | 0.43 | 0.42 | 0.40 |

TABLE 8

| | | Delivered Dose Uniformity (mg/actuation) | | | |
|---|---|---|---|---|---|
| | Through Unit | BESPAK Valve/ | | APTAR Valve/ | |
| | Life Test | FEP Coated Canister | | FEP Coated Canister | |
| Composition | Position | 0 Week | 13 Weeks | 0 Week | 13 Weeks |
| HFA-134a | Beginning | 19.0 | 18.4 | 18.2 | 17.8 |
| | Middle | 18.2 | 19.3 | 17.8 | 18.2 |
| | End | 19.5 | 19.8 | 18.1 | 18.1 |
| | Mean | 18.9 | 19.2 | 18.0 | 18.1 |
| HFA-152a | Beginning | 17.1 | 19.7 | 16.9 | 18.0 |
| | Middle | 16.9 | 19.1 | 16.4 | 17.6 |
| | End | 17.6 | 20.0 | 17.0 | 18.1 |
| | Mean | 17.2 | 19.6 | 16.8 | 17.9 |
| HFO-1234ze(E) | Beginning | 17.1 | 18.3 | 16.5 | 17.0 |
| | Middle | 15.1 | 16.7 | 16.1 | 16.9 |
| | End | 15.5 | 18.0 | 16.3 | 16.8 |
| | Mean | 15.9 | 17.7 | 16.3 | 16.9 |

TABLE 9

| | | Maximum Total | | |
|---|---|---|---|---|
| | | Impurities (% w/w) | | |
| Composition | Valve/Canister | 0 Week | 6 Weeks | 13 Weeks |
| HFA-134a | BESPAK/Aluminium | ND | ND | 2.60 |
| | BESPAK/Stainless Steel | ND | 0.35 | 0.99 |
| | BESPAK/FEP Coated | ND | 0.55 | 1.49 |
| | APTAR/FEP Coated | ND | 0.49 | ND |
| HFA-152a | BESPAK/Aluminium | 2.32 | 2.11 | 2.39 |
| | BESPAK/Stainless Steel | 2.36 | 2.59 | 2.94 |
| | BESPAK/FEP Coated | 2.32 | 2.18 | 2.33 |
| | APTAR/FEP Coated | 2.31 | 3.24 | 2.13 |
| HFO-1234ze(E) | BESPAK/Aluminium | 0.18 | 0.22 | 1.25 |
| | BESPAK/Stainless Steel | 0.22 | 0.51 | 0.49 |
| | BESPAK/FEP Coated | 0.20 | 1.10 | 1.19 |
| | APTAR/FEP Coated | 0.21 | 0.76 | 0.13 |

It was observed that in all configurations/compositions tested, ipratropium content remained consistent and at anticipated levels at all timepoints

It was also observed that in all configurations/compositions tested, through unit life delivered dose remained consistent and at anticipated levels at all timepoints.

It was also observed that in all configurations/compositions tested, maximum total impurities were higher in HFA-152a compositions, when compared to HFA-134a and HFO-1234ze(E). It was also observed that for each composition there is an influence of valve and canister configuration on maximum total impurities, with the exception of HFA-152a compositions, which display higher and more consistent maximum total impurities regardless of valve and canister configuration. It was also observed that after 13 wks storage at 40° C. and 75% relative humidity, relatively low total impurities were present in HFO-1234ze(E) when compared to corresponding HFA-152a compositions.

It was learned that equivalent ipratropium bromide solution compositions in HFO-1234ze(E) and HFA-152a gave comparable and consistent ipratropium bromide content and through unit life delivered dose, and also demonstrated similarity to HFA-134a composition results when tested after 13 weeks storage at 40° C. and 75% relative humidity. It was also learned that HFO-1234ze(E) compositions gave lower maximum total impurities when tested after 13 weeks storage at 40° C. and 75% relative humidity when compared to equivalent HFA-152a compositions with comparable formulation components, and the same valves and canisters.

### Example 8: Solution Formulations Containing Glycopyrronium Bromide and HFO-1234ze(E)

Four solution formulations containing glycopyrronium bromide in HFO-1234ze were prepared as described in Table 10 (formulation composition listed in % w/w). The formulations were prepared and filled into clear vials to enable visual observation of the formulation to assess if the drugs were dissolved in solution. The formulations were stored at refrigerated conditions (approximately 5° C.). At various timepoints, the formulations were removed from refrigeration and the formulations were inspected to assess if the solutions remained clear. The vials were returned to refrigerated conditions between observations. The appearance of the formulations at various timepoints post-manufacturing is described in Table 11.

It was observed that formulations with 12% ethanol by weight and 0.024% 1 M HCl by weight were not stable solutions for formulations containing only glycopyrronium bromide (Formulation D) or containing a combination of all three drugs (Formulation A). However, formulations with 15% or 18% ethanol by weight and 1 M HCl of 0.030% and 0.036% by weight, respectively, remained as clear, and therefore, physically stable solutions for more than 70 days when stored at refrigerated conditions (approximately 5° C.).

From this example, it was learned that glycopyrronium bromide was soluble in HFO-1234ze(E) with either 15% or 18% ethanol by weight when also including approximately 0.03% 1 M hydrochloric acid by weight.

TABLE 10

| Formulation | Formulation | Formulation | Formulation | Formulation |
|---|---|---|---|---|
| Constituents | A | B | C | D |
| Beclomethasone | 0.175% | 0.176% | 0.175% | 0.000% |
| Dipropionate | | | | |
| Formoterol | 0.010% | 0.010% | 0.010% | 0.000% |
| Fumarate | | | | |
| Dihydrate | | | | |
| Glycopyrronium | 0.023% | 0.024% | 0.023% | 0.023% |
| Bromide | | | | |
| Anhydrous | 12.008% | 15.014% | 17.976% | 12.075% |
| Ethanol | | | | |
| 1M Hydrochloric | 0.024% | 0.030% | 0.036% | 0.024% |
| Acid | | | | |
| HFO-1234ze(E) | 87.759% | 84.746% | 81.779% | 87.877% |

TABLE 11

| Time Post- | | | | |
|---|---|---|---|---|
| Manufacturing | Formulation | Formulation | Formulation | Formulation |
| (days) | A | B | C | D |
| 0 | Clear | Clear | Clear | Clear |
| | solution | solution | solution | solution |
| 1 | Clear | Clear | Clear | Clear |
| | solution | solution | solution | solution |
| 2 | Clear | Not | Not | Clear |
| | solution | Measured | Measured | solution |
| 4 | Not | Clear | Clear | Not |
| | Measured | solution | solution | Measured |
| 5 | Fine | Not | Not | Clear |
| | particles | Measured | Measured | solution |
| | observed in | | | |
| | formulation | | | |
| 71 | Not | Clear | Clear | Not |
| | Measured | solution | solution | Measured |
| 72 | Fine | Not | Not | Crystals |
| | particles | Measured | Measured | observed |
| | observed | | | settled |
| | in | | | on bottom |
| | formulation | | | of |
| | | | | formulation |

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A composition comprising:
a solution comprising:
an active pharmaceutical ingredient comprising an anticholinergic agent; and
a propellant comprising HFA-152a, HFO-1234ze(E), or both.

2. The composition of claim 1, wherein the anticholinergic agent comprises a long-acting muscarinic antagonist.

3. The composition of claim 1, wherein the anticholinergic agent is selected from ipratropium, tiotropium, aclidinium, umeclidinium, glycopyrronium, a pharmaceutically acceptable salt or ester of any of the listed drugs, or a mixture of any of the listed drugs, their pharmaceutically acceptable salts or their pharmaceutically acceptable esters.

4. The composition of claim 1, wherein the anticholinergic agent comprises a quaternary ammonium salt.

5. The composition of claim 1, wherein the anticholinergic agent is selected from ipratropium, tiotropium, glycopyrronium, and a pharmaceutically acceptable salt or ester of any of the listed drugs.

6. A composition comprising:
a solution comprising:
ipratropium bromide; and
HFA-152a, HFO-1234ze(E), or both.

7. The composition of claim 1, wherein the active pharmaceutical ingredient consists essentially of ipratropium bromide.

8. The composition of claim 1, the solution further comprising ethanol.

9. The composition of claim 8, wherein ethanol is present at 5 wt-% to 22.5 wt-% of the total composition.

10. The composition of claim 1, the solution further comprising an acid.

11. The composition of claim 1, the solution further comprising water.

12. The composition of claim 10, wherein the acid is present at a concentration of 0.001 wt-% to 5 wt-% of the total composition.

13. The composition of claim 11, wherein water is present at 0.1 wt-% to 1 wt-% of the total composition.

14. The composition of claim 1, wherein HFA-152a, HFO-1234ze(E), or a combination thereof makes up 75 wt-% to 95 wt-% of the total composition.

15. The composition of claim 1, wherein the active pharmaceutical ingredient is present at 0.02 wt-% to 0.06 wt-% of the total composition.

16. A composition comprising:
a solution comprising:
ipratropium bromide;
HFA-152a, HFO-1234ze(E), or both;
an acid;
water; and
ethanol.

17. A metered dose inhaler comprising:
a metering valve;
a canister; and
an actuator comprising an actuator nozzle;
wherein the canister comprises the composition of claim 1.

18. The metered dose inhaler of claim 17, wherein the metering valve comprises a metering chamber having a size between 25 microliters and 200 microliters.

19. The metered dose inhaler of claim 17, wherein the actuator has an exit orifice diameter between 0.12 mm and 0.4 mm.

20. The metered dose inhaler of claim 17, wherein the canister comprises from about 1 mL to about 30 mL of the composition.
